(19) United States
(12) Patent Application Publication
(10) Pub. No.: US 2003/0113901 A1
(43) Pub. Date: Jun. 19, 2003

(54) Cell production

(76) Inventors: Robert Anthony Hirst, Norwood (AU); Joy Rathjen, Blackwood (AU); Peter David Rathjen, Blackwood (AU)

Correspondence Address: William L. Warren, SUTHERLAND ASBILL & BRENNAN LLP, 999 Peachtree Street, NE, Atlanta, GA, 30309-3996, US

(21) Appl. No.: 10/045,963

(22) Filed: Oct. 24, 2001

(30) Foreign Application Priority Data
Oct. 24, 2000 (AU) PR0952

(51) Int. Cl. C12N 1/20

(52) U.S. Cl. 435/252.3

(57) ABSTRACT

A method for producing differentiated or partially differentiated neurons which method includes providing

a source of neurectoderm cells which are isolated or otherwise derived from sources other than adult or foetal brain tissue;

a culture medium; and

a growth factor from the neural growth factor (NGF) family; culturing the cells in the culture medium and in the presence or absence of the NGF growth factor to produce differentiated or partially differentiated neurons.

The present invention relates to differentiated or partially differentiated neuronal cells derived from neurectoderm and particularly dopaminergic neuronal cells. The present invention also relates to methods of producing and culturing the cells of the present invention, and to uses thereof.

Pluripotent cells can be isolated from the pre-implantation mouse embryo as embryonic stem (ES) cells. ES cells can be maintained indefinitely as a pluripotent cell population in vitro, and, when reintroduced into a host blastocyst, can contribute to all adult tissues of the mouse including the germ cells. ES cells, therefore, retain the ability to respond to all the signals that regulate normal mouse development, and potentially represent a powerful model system for the investigation of mechanisms underlying pluripotent cell biology and differentiation within the early embryo, as well as providing opportunities for embryo manipulation and resultant commercial, medical and agricultural applications. Other pluripotent cells and cell lines will share some or all of these properties and applications.

The successful isolation long term clonal maintenance, genetic manipulation and germ-line transmission of pluripotent cells from species other than rodents has generally been difficult to date and the reasons for this are unknown. International patent application WO97/32033 and U.S. Pat. No. 5,453,357 describe pluripotent cells including cells from species other than rodents, and primate pluripotent cells have been described in International patent applications WO98/43679 and WO96/23362 and in U.S. Pat. No. 5,843,780.

The differentiation of ES cells can be regulated in vitro by the cytokine leukaemia inhibitory factor (LIF) and other gp130 agonists which promote self-renewal and prevent differentiation of the stem cells. However, there is little information about biological molecules that can induce the differentiation of ES cells into specific cell types. Chemical inducers such as retinoic acid have been used to form limited neural lineages. However, the variety of neural cell types produced by these methods is limited.

Neurons are functionally important nerve cells because they are responsible for the production and recognition of chemical signals in the brain and central nervous system (CNS). A sub-class of neurons, termed dopaminergic (DA) neurons produce and secrete neurotransmitter dopamine. It is the degeneration of these dopaminergic neurons in an area of the brain called the Substantial Nigra that is a major cause of Parkinson's Disease.

The treatment of neurodegenerative and CNS disorders using cell replacement will require the production and maintenance of neurons, including DA neurons.

Derivation of dopaminergic neurons has been described in U.S. Pat. No. 5,981,165. These cells were derived from neural stem cells isolated from the central nervous system, and differentiation was induced by culture on astrocyte feeder cells, in the presence of FGF, with a TGFβ family member and/or feeder cells and/or conditioned medium. The differentiation procedure does not generate essentially homogeneous populations of dopaminergic neurons. Mixed cell populations are not optimal for use in the treatment of neurodegenerative diseases such as Parkinson's disease.

The production of midbrain and hindbrain neurons, including dopaminergic neurons, from mouse ES cells in vitro has also been reported (Lee et al., 2000). However, there were difficulties in directing efficient differentiation of ES cells such that essentially homogeneous populations of neuronal cells were formed, leading to further difficulties in producing cell populations comprising predominantly dopaminergic neurons. In particular a maximum of about 20% of cells produced by Lee et al were dopaminergic. It is an object of the present invention to overcome, or at least alleviate, one or more of the difficulties or deficiencies associated with the prior art.

Applicant has surprisingly found that pluripotent cell differentiation can be regulated to produce essentially homogeneous populations of neurectoderm cells, and that differentiation of neurectoderm can be further regulated to form cells of the neural, glial and neural crest lineages. These findings are outlined in International patent application PCT/AU01/00030 “Cell Production” filed by applicants, the entire disclosure of which is incorporated herein by reference.

In a first aspect of the present invention there is provided a method for producing differentiated or partially differentiated neurons which method includes

providing

a source of neurectoderm cells;

a culture medium; and

a growth factor from the neural growth factor (NGF) family;

culturing the cells in the culture medium and in the presence or absence of the NGF growth factor to produce differentiated or partially differentiated neurons.

Applicant has found that neural growth factor (NGF) when included in culture with neurectoderm cells, preferably In vitro-derived neurectoderm cells, induces the formation of cell populations consisting predominantly of neurons. These neurons were identified morphologically by the presence of axonal projections, and stained positive for a known neuronal marker, NF 200. Only a small proportion of these neurons were identified as dopaminerglo.

As used herein, the term “neurectoderm cell” refers to undifferentiated neural progenitor cells substantially equivalent to cell populations comprising the neural plate and/or neural tube and isolated or otherwise derived from sources other than adult or foetal brain tissue. They are multipotential, and have the capacity to differentiate into all cell types that make up the central nervous system and peripheral nervous system, including neuronal cells, glial cells and neural crest cells (e.g. as outlined in International patent application PCT/AU01/00030 “Cell Production” to applicants, the entire disclosure of which is incorporated herein by reference).

The neurectoderm cells may be in vitro-derived neurectoderm cells. The neurectoderm cells may be characterised in that they are:

essentially homogeneous;

able to differentiate on to all neural cell types neurons, glia cells and neural crest cells); and

unpatterned.

The neurectoderm cells may include cells derived from early primitive ectoderm-like (EPL) cells, e.g. according to the method described in International patent application PCT/AU01/00030, above.

The neurectoderm cells so produced may be utilised in the production of partially or terminally differentiated neural cells as described above.

In a preferred embodiment, the culturing step may produce a heterogeneous cell population including neurons, the method may include, subsequent to the culturing step,

selecting neurons from the heterogeneous cell population.

The method of selection may utilise any standard methods including utilisation of suitable markers for neural cells, including cell surface markers and gene expression markers and morphology. Known markers such as the neuronal marker NF200 may be used.

In a particular preferred embodiment of the present invention, there is provided a method for producing dopaminergic (DA) neurons, the degeneration of which, as stated above, are implicated in the development of Parkinson's Disease.

Accordingly in this embodiment, there is provided a method for producing dopaminergic (DA) neurons, which method includes

providing

a source of neurectoderm cells;

a culture medium;

a growth factor from the neural growth factor (NGF) family; and

a source of dopamine; and

culturing the cells in the presence of the culture medium and in the presence of the NGF growth factor and dopamine to produce differentiated or partially differentiated dopaminergic (DA) neurons.

Applicant has found that when neurectoderm cells, preferably in vitro-derived neurectoderm cells, are incubated in the presence of NGF and dopamine, the resulting cell population contains a significant amount of cells that stained positive for TH, an enzyme essential for the biosynthesis of dopamine in neurons. These TH-positive cells resembled nerve cells morphologically, including the presence of axon-like projections.

The dopaminergic biosynthetic pathway is part of a general biosynthetic pathway used for the production of all catecholamine neurotransmitters, including dopamine, norepinephrine and epinephrine. In some of these neurons this pathway is terminated with the production of dopamine. In other catecholaminergic neurons the biosynthetic pathway is extended, and dopamine is converted into norepinephrine and epinephrine. Some catecholaminergic neurons have the potential to synthesize all three catecholamines.

These results clearly show that in the presence of NGF, in vitro-derived neurectoderm can be directed to form cell populations consisting predominantly of neurons. Furthermore, in the presence of NGF and dopamine, in vitro derived neurectoderm can be directed to produce cell populations comprised predominantly of neurons expressing TH.

In summary neurectoderm differentiation has been directed to form cell populations enriched in dopaminergic neurons. Furthermore neurons so formed may have the capacity to synthesise other catecholamine neurotransmitters.

In a further aspect of the present invention there is provided a partially or terminally differentiated neuronal cell, produced by the methods according to the present invention.

The differentiated or partially differentiated neuronal cell derived herein may be from any vertebrate including murine, human, bovine, ovine, porcine, caprine, equine and chicken.

The neurectoderm cells may be cultured according to the present inventions under conditions suitable for their proliferation and maintenance in vitro. This includes the use of serum including foetal calf serum and bovine serum or the medium may be serum-free. Other growth enhancing components such as insulin, transferrin and sodium selenite may be added to improve growth of the cells from which the conditioned medium or extracellular matrix may be derived. As would be readily apparent to a person skilled in the art, the growth enhancing components will be dependent upon the cell types cultured, other growth factors present, attachment factors and amounts of serum present.

The neurectoderm cells may be cultured for a time sufficient to establish the cells in culture. By this we mean a time when the cells equilibrate in the culture medium. Preferably the cells are cultured for approximately 3-5 days.

The neurectoderm cell culture medium may be any cell culture medium appropriate to sustain the cells employed. The culture medium may be a conditioned medium generated from a liver cell or liver cell line, preferably using Dulbecco's Modified Eagles Medium (DMEM) containing high glucose, supplemented with 10% FCS, 40 μg/ml gentamycin, 1 mM L-glutamine.

Separation of the conditioned medium from the cells may be achieved by any suitable technique, such as decanting the medium from the cells. Preferably the cell culture medium is clarified by centrifugation or filtration (e.g. through a 0.22 μM filter) to remove excess cells and cellular debris. Other known means of separating the cells from the medium may be employed providing the separation method does not remove the growth components from the medium.

In a further aspect of the present invention, there is provided a method for the production of genetically modified neuronal cells, said method including

providing

a source of genetically modified neurectoderm cells;

a culture medium;

a growth factor from the neural growth factor (NGF) family;

culturing the cells in the culture medium and in the presence or absence of the NGF growth factor to produce differentiated or partially differentiated genetically modified neurons.

Modification of the genes of these cells may be conducted by any means known to the skilled person which includes introducing extraneous DNA, removing DNA or causing mutations within the DNA of these cells. Modification of the genes includes any changes to the genetic make-up of the cell thereby resulting in a cell genetically different to the original cell.

In a further aspect of the present invention there is provided a partially or terminally differentiated genetically modified neuron, produced by the methods of the present invention.

The neuronal cells of the present invention and the differentiated or partially differentiated cells derived therefrom, have a number of uses, including the following:

in human cell therapy to treat neuronal diseases. For example neuronal cells including neurons and dopaminergic (DA) neurons may be used in cell therapy to treat Parkinson's disease.

in human gene therapy to treat neuronal diseases

to produce cells, tissues or components of organs for transplant

as cytoplasts or karyoplasts in nuclear transfer

as a source of nuclear material for nuclear transfer

Accordingly, in a preferred aspect of the present invention, there is provided a method for the treatment of neurodegenerative and central nervous system (CNS) disorders in mammals, including humans, which method includes utilising the neuronal cells described above in cell replacement therapies.

The neuronal cells utilised in the method according to the present invention may include neurectoderm cells, or neurons including dopaminergic (DA) neurons.

The present invention will now be more fully described with reference to the accompanying examples and drawings. It should be understood, however, that the description following is illustrative only and should not be taken in any way as a restriction on the generality of the invention described above.

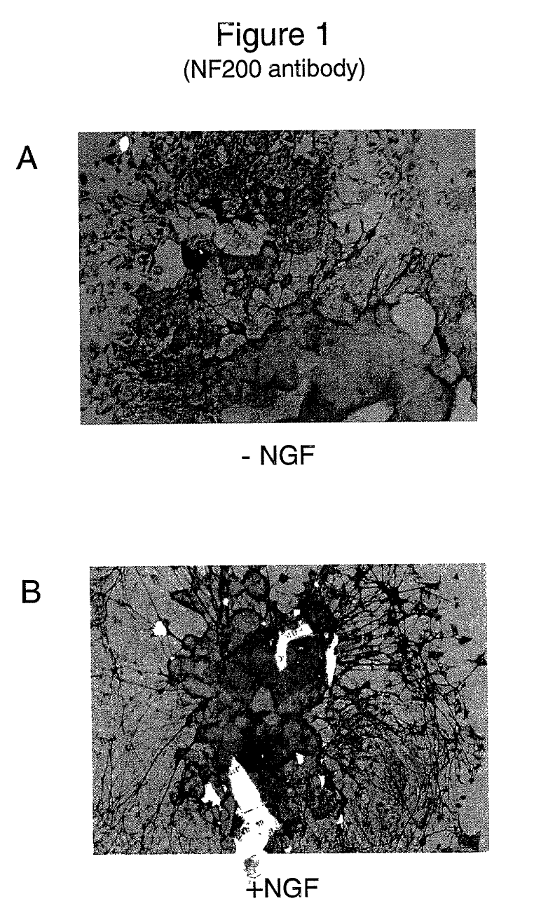

FIG. 1: The culture of in vitro-derived neurectoderm in the presence of NGF increases neuron formation. EBM⁷s were seeded for 3 days in 50% DMEM/F12, 10 ng/ml FGF2, 0.1% ITSS, 1% FCS, in the presence or absence of 10 ng/ml NGF, and for an additional 4 days in 50% DMEM/F12, 0.1% ITSS. The resulting EBMs were analysed by immunohistochemistry using an anti-NF200 antibody. Positive staining for NF200 (dark regions) indicated neurons. A, EBM⁷s cultured in the absence of NGF; B, EBM⁷s cultured in the presence of NGF.

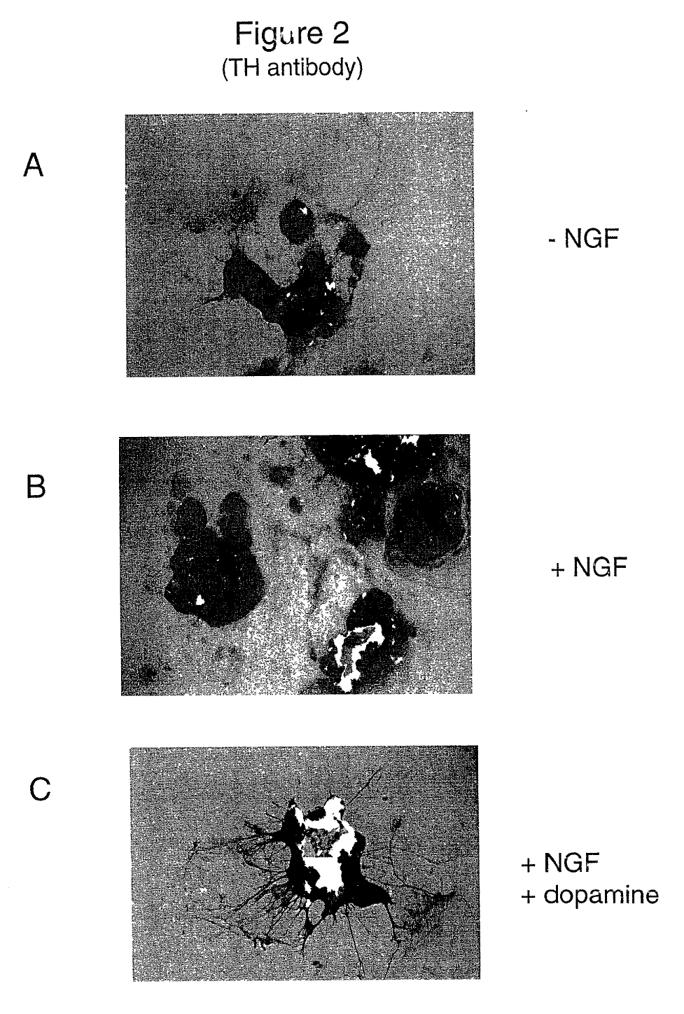

FIG. 2: The culture of in vitro-derived neurectoderm in the presence of NGF and dopamine increases dopaminergic neuron formation. EBM⁷s were seeded for 3 days in 50% DMEM/F12, 10 ng/ml FGF2, 0.1% ITSS, 1% FCS, and 10 ng/ml NGF, and for an additional 4 days in DMEM/F12, 0.1% ITSS, 200 μM ascorbic acid, with or without 200 μM dopamine. The resulting EBMs were analysed by immunohistochemistry using an anti-TH antibody. Positive staining for TH (dark regions) indicated dopaminergic neurons. A, EBM⁷s cultured in the absence of NGF and dopamine; B, EBM⁷s cultured in the presence of NGF; C, EBM⁷s cultured in the presence of NGF and dopamine.

### EXAMPLE 1

### The production of neurons, including dopaminergic neurons, from in vitro-derived neurectoderm

### Methods:

Formation of neurectoderm

EBs were grown in the presence of the conditioned medium MEDII for 7 days (EBM⁷) as previously described in International Patent Application PCT/AU99/00265 to applicants, the entire disclosure of which is incorporated herein by reference.

ES cells were seeded at a density of 1×10⁵ cells/cm² In suspension culture in bacterial petri dishes in DMEM containing 50% MEDII. DMEM is defined as Dulbecco's Modified Eagles Medium (Gibco BRL), pH 7.4, containing high glucose and supplemented with 10% foetal calf serum (FCS; Commonwealth Serum Laboratories), 40 μg/ml gentamycin, 1 mM L-glutamine, 0.1 mM β-mercaptoethanol (β-ME). The resulting cell aggregates (EBMs) were split 1:2 after 2 days and seeded into fresh DMEM containing 50% MEDII.

Neurectderm derived in vitro has the capacity to form cell populations that consist predominantly of neurons or dopaminergic neurons

On day 7, EBM⁷s were seeded in 50% DMEM/50% F12 supplemented with 10 ng/ml FGF2 (Sigma), 0.1% ITSS (Boehringer Mannheim), 1% FCS in the presence or absence of 10 ng/ml NGF. On day 10, the medium was changed to fresh 50% DMEM/50% F12 containing 0.1% ITSS, 200 μM ascorbic acid, with or without 200 μM dopamine (Sigma, H8502, Lot 19H252) for a further 4 days, with daily replenishment of the medium. On day 14 in preparation for in situ hybridisation, cells were fixed in 4% paraformaldehyde in PBS, and blocked in the appropriate blocking buffer for 30 minutes followed by overnight incubation with primary antibodies in blocking buffer at 4° C. Aggregates were washed with PBS (3×5 minutes) before incubation with species-specific secondary antibodies in blocking buffer for 1 hour. After washing with PBS (3×5 minutes) the plates were developed and examined on a Nikon TE300 microscope.

Antibody staining for NF200 and tyrosine hydroxylase (TH) was detected by alkaline phoaphatase activity.

NF200: Primary antibody: anti-neurofilament 200 (Sigma immunochemicals N-4142). Secondary antibody: Alkaline Phosphatase [Goat] Anti-Rabbit IgG (H & L) conjugate (Zymed Laboratories Inc., USA).

Tyrosine Hydroxylase Primary antibody: monoclonal Anti-Tyrosine Hydroxylase (mouse IgG isotype) as a primary antibody. Secondary antibody: Alkaline Phosphatase conjugated [Goat] anti-mouse IgG (Rockland, USA).

### Results:

### Immunohistochemistry, NF200:

The results for the effect of the addition of NGF to neurectoderm are shown in FIG. 1.

In the absence of NGF, differentiation of EBM resulted in the formation of a heterogeneous cell population that contained a small proportion of cells staining positive for NF200, a known marker for neurons. Morphologically, the NF200 positive cells closely resembled neurons, as indicated by the presence of axon-like projections. When EBM⁷s were grown in the presence of NGF, the proportion of cells staining positive for NF200 was significantly increased. As before, the cells staining positive for NF200 closely resembled neurons.

### Immunohistochemistry, TH:

The results for the addition of dopamine to differentiating neurectoderm are shown in FIG. 2.

EBMs grown in the absence of dopamine, with or without NGF, contained only a small proportion of TH positive cells.

Growth of EBMs in the presence of dopamine resulted in a significant increase in cells that stained positive with anti-TH antibody. These TH-positive cells resembled neurons morphologically, as indicated by the presence of axon-like projections. Numbers of TH-positive cell numbers were maximal in the presence of NGF and dopamine.

In differentiated cells dopamine inhibits tyrosine hydroxylase activity (Ganong WF. Review of Medical Physiology. (Appleton & Lange publishers). The results observed here are not consistent with inhibitory action. In these experiments the cells were not terminally differentiated, but instead were early neural progenitor cells. Hence dopamine appears to exert different effects on partially differentiated neural cells, including upregulation of tyrosine hydroxylase expression, and directed differentiation towards derivation of dopaminergic cells. Such action is consistent with the widely reported upregulation of neurotransmitter receptors in response to increased neurotransmitter levels from low levels (eg., Ulrich et al (1997).

It is possible that the mechanism where dopamine promotes the production of dopaminergic neurons from neural progenitor cells is a common mechanism for the terminal differentiation of neurons in different brain regions with other neurotransmitter environments.

### REFERENCES

Ganong WF. Review of Medical Physiology. (Appleton & Lange publishers)

Sang-Hun Lee, Nadya Lumelsky, Lorenz Studer, Jonathan M. Auerbach, and Ron D. McKay. Efficient generation of midbrain and hindbrain neurons from mouse embryonic stem cells. Nature Biotechnology 18, pp 675-679.

Ulrich Y M, Hargreaves K M and Flores C M (1997). A comparison of multiple injections versus continuous fusion of nicotine for producing upregulation of neuronal [H-3]-epibatidine binding sites. Neuropharmacology 36, 1119-1125.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

1. A method for producing differentiated or partially differentiated neurons which method includes
providing
a source of neurectoderm cells which are isolated or otherwise derived from sources other than adult or foetal brain tissue;
a culture medium; and
a growth factor from the neural growth factor (NGF) family;
culturing the cells in the culture medium and in the presence or absence of the NGF growth factor to produce differentiated or partially differentiated neurons.

2. A method according to claim 1, wherein the culturing step is conducted in the presence of the NGF growth factor.

3. A method according to claim 1, wherein the neurectoderm cells are in vitro derived neurectoderm cells.

4. A method according to claim 3, wherein the neurectoderm cells are characterised in that they are
essentially homogeneous;
able to differentiate on to all neural cell types; and
unpatterned.

5. A method according to claim 4, wherein the neurectoderm cells are derived from differentiation of early primitive ectoderm-like (EPL) cells.

6. A method according to claim 5, wherein the culturing step produces a heterogeneous cell population including neurons, the method further including subsequent to the culturing step,
selecting neurons from the heterogeneous cell population.

7. A method according to claim 6, wherein the selection method utilises markers for neural cells, including cell surface markers, gene expression markers and morphology.

8. A method for producing dopaminergic (DA) neurons, which method includes
providing
a source of neurectoderm cells which are isolated or otherwise derived from sources other than adult or foetal brain tissue;
a culture medium;
a growth factor from the neural growth factor (NGF) family; and
a source of dopamine; and
culturing the cells in the presence of the culture medium and in the presence of the NGF growth factor and dopamine to produce differentiated or partially differentiated dopaminergic (DA) neurons.

9. A method according to claim 8, wherein the neurectoderm cells are in vitro derived neurectoderm cells.

10. A method according to claim 9, wherein the neurectoderm cells are derived from differentiation of early primitive ectoderm-like (EPL) cells.

11. A method according to claim 10, wherein the resulting cell population contains a significant amount of cells that stain positive for TH.

12. A method according to claim 11, wherein the TH positive cells resemble nerve cells morphologically, including the presence of axon-like projections.

13. A method according to claim 8, wherein the culture medium is a conditioned medium including Dulbecco's Modified Eagles Medium (DMEM) and foetal calf serum (FCS).

14. A method according to claim 13, wherein the culture medium further includes FGF2 and exhibits a high glucose content.

15. A method according to claim 8, further including the step of separating the cells from the conditioned medium.

16. A method according to claim 15, wherein the separation step includes decanting, centrifugation or filtration, or a combination thereof.

17. A partially or terminally differentiated neuronal cell, whenever produced by a method according to any one of the preceding claims.

18. A partially or terminally differentiated neuronal cell according to claim 17, derived from a vertebrate selected from the group consisting of murine, human, bovine, ovine, porcine, caprine, equine and chicken.

19. A partially or terminally differentiated neuronal cell according to claim 18, wherein the cells are dopaminergic (DA) neurons.

20. A partially or terminally differentiated neuronal cell according to claim 18, wherein the cells are TH positive and resemble nerve cells morphologically, including the presence of axon-like projections.

21. A method for the production of genetically modified neuronal cells, said method including
providing
a source of genetically modified neurectoderm cells;
a culture medium;
a growth factor from the neural growth factor (NGF) family;
culturing the cells in the culture medium and in the presence or absence of the NGF growth factor to produce differentiated or partially differentiated genetically modified neurons.

22. A method according to claim 21, wherein the culturing step is conducted in the presence of the NGF growth factor.

23. A method according to claim 22, wherein the neurectoderm cells are in vitro derived neurectoderm cells.

24. A method according to claim 23, wherein the neurectoderm cells are derived from differentiation of early primitive ectoderm-like (EPL) cells.

25. A method according to claim 22, wherein the modification of the genes includes introducing extraneous DNA, removing DNA, or causing mutations within the DNA of the cells.

26. A method according to claim 21, wherein the culturing step is conducted in the presence of the NGF growth factor and dopamine, to produce differentiated or partially differentiated genetically modified dopaminergic (DA) neurons.

27. A method according to claim 26, wherein the resulting cell population contains a significant amount of cells that stain positive for TH and resembled nerve cells morphologically, including the presence of axon-like projections.

28. Use of genetically modified or unmodified neuronal cells produced according to any one of claims 1 to 16 and 21 to 27, or their differentiated or partially differentiated progeny, for use in human cell therapy or transgenic animal production.

29. Use of genetically modified or unmodified neuronal cells produced according to any one of claims 1 to 16 and 21 to 27, or their differentiated or partially differentiated progeny, for use in human or animal gene therapy.

30. A method for the treatment of neurodegenerative and central nervous system (CNS) disorders in mammals, including humans, which method includes utilising the neuronal cells produced according to any one of claims 1 to 16 and 21 to 27 in cell replacement therapies.

31. A method according to claim 30, wherein the neuronal cells include dopaminergic (DA) neurons.
